(19) United States
(12) Patent Application Publication
(10) Pub. No.: US 2026/0294387 A1
(43) Pub. Date: Oct. 1, 2026

(54) STEERING SYSTEM FOR ULTRASOUND CATHETERS

(71) Applicant: GE Precision Healthcare LLC, Waukesha, WI (US)

(72) Inventor: Hani Nozari MIRAR, Oslo (NO)

(21) Appl. No.: 19/089,868

(22) Filed: Mar. 25, 2025

(51) Int. Cl. A61B 8/12; A61B 8/00; A61B 8/06; A61B 34/30

(52) U.S. Cl. CPC A61B 8/12; A61B 8/06; A61B 8/461; A61B 8/5223; A61B 34/30; A61B 2034/301

(57) ABSTRACT

A steering system may receive a selection of an anatomical structure of interest of a subject and a quantification task associated with the anatomical structure of interest of the subject. The steering system may determine a current orientation of an ultrasound catheter in the subject. The steering system may determine a target orientation of the ultrasound catheter in the subject for the quantification task associated with the anatomical structure of interest of the subject. The steering system may determine steering information for steering the ultrasound catheter in the subject from the current orientation to the target orientation. The steering system may perform an action based on the steering information.

### TECHNICAL FIELD

The present disclosure relates to a steering system for ultrasound catheters. More specifically, the present disclosure relates to a steering system for ultrasound catheters that determines steering information for steering an ultrasound catheter in a subject from a current orientation to a target orientation for a quantification task associated with an anatomical structure of interest of the subject.

### BACKGROUND

An ultrasound catheter is a device that is introduced into the body of a subject and that is configured to acquire ultrasound data from within the body of the subject. For example, intracardiac echocardiography (ICE) utilizes an ultrasound catheter to perform imaging of the heart from within the heart. For ICE, an ultrasound catheter is inserted into the body of the subject through, typically, the femoral artery and is advanced into the heart of the subject. ICE may be used for invasive procedures such as mitral valve repair, atrial septal defect closure, left heart ablations, or the like.

An ultrasound catheter generally includes a transducer that is configured to acquire ultrasound data. For example, the transducer may be positioned at a tip of the ultrasound catheter, adjacent to a tip of the ultrasound catheter, to a side of the tip of the ultrasound catheter, or the like. Further, an ultrasound catheter generally includes one or more steering components (e.g., a knob, a handle, a dial, or the like) that permit the transducer to be steered. For example, a steering component may steer the tip by adjusting rotation of the transducer about a yaw axis, rotation of the transducer about a pitch axis, rotation of the transducer about a roll axis, or translation of the transducer about a longitudinal axis (or roll axis). A user of the ultrasound catheter may steer the ultrasound catheter by manipulating the one or more steering components.

An ultrasound catheter may be used for performing a quantification task associated with an anatomical structure of interest. For example, a quantification task may be a measurement of a blood flow parameter, such as a measurement of a blood flow direction through the anatomical structure of interest, a measurement of a blood flow velocity through the anatomical structure of interest, a measurement of a blood flow rate through the anatomical structure of interest, or the like. Additionally, or alternatively, the quantification task may be a measurement of a dimension of the anatomical structure of interest, or the like. In some cases, the ultrasound catheter may be used for performing a set of quantification tasks associated with a set of anatomical structures as specified by a protocol.

Generally, the orientation of an ultrasound catheter relative to an anatomical structure of interest may affect the quality, or accuracy, of the quantification task. For instance, an ultrasound system may determine a blood flow direction and/or a blood flow direction through an anatomical structure of interest based on a transmit frequency of ultrasound signals, a received frequency of echo signals, and an angle of insonation between an ultrasound beam of the ultrasound catheter and the direction of blood flow in the anatomical structure of interest. Further, the ultrasound system may determine a blood flow velocity based on a frequency shift between the transmitted ultrasound signals and the received echo signals and the insonation angle. The determined blood flow velocity may, or might not, be accurate based on the particular angle of insonation between the ultrasound beam of the ultrasound catheter and the direction of blood flow. For example, an insonation angle of 0° may result in an accurate blood velocity measurement, whereas insonation angles greater than 60° may result in relatively inaccurate blood flow velocity measurements.

### SUMMARY

This summary introduces concepts that are described in more detail in the detailed description. It should not be used to identify essential features of the claimed subject matter, nor to limit the scope of the claimed subject matter.

In an aspect, a system may include a memory configured to store instructions; and one or more processors configured to execute the instructions to: receive a selection of an anatomical structure of interest of a subject and a quantification task associated with the anatomical structure of interest of the subject; determine a current orientation of an ultrasound catheter in the subject; determine a target orientation of the ultrasound catheter in the subject for the quantification task associated with the anatomical structure of interest of the subject; determine steering information for steering the ultrasound catheter in the subject from the current orientation to the target orientation; and perform an action based on the steering information.

In another aspect, a method may include receiving a selection of an anatomical structure of interest of a subject and a quantification task associated with the anatomical structure of interest of the subject; determining a current orientation of an ultrasound catheter in the subject; determining a target orientation of the ultrasound catheter in the subject for the quantification task associated with the anatomical structure of interest of the subject; determining steering information for steering the ultrasound catheter in the subject from the current orientation to the target orientation; and performing an action based on the steering information

In yet another aspect, a non-transitory computer-readable medium may store instructions that, when executed by one or more processors, cause the one or more processors to: receive a selection of an anatomical structure of interest of a subject and a quantification task associated with the anatomical structure of interest of the subject; determine a current orientation of an ultrasound catheter in the subject; determine a target orientation of the ultrasound catheter in the subject for the quantification task associated with the anatomical structure of interest of the subject; determine steering information for steering the ultrasound catheter in the subject from the current orientation to the target orientation; and perform an action based on the steering information.

### BRIEF DESCRIPTION OF DRAWINGS

FIG. 1 is a diagram of an example system for steering an ultrasound catheter in a subject from a current orientation to a target orientation for a quantification task associated with an anatomical structure of interest of the subject.

FIG. 2 is a diagram of an example steering system of FIG. 1.

FIG. 3 is a diagram of an example ultrasound system of FIG. 1.

FIG. 4 is a diagram of an example ultrasound catheter of the ultrasound system of FIG. 3.

FIG. 5 is a diagram of an example tracking system of FIG. 1.

FIG. 6 is a diagram of an example preoperative imaging system of FIG. 1.

FIG. 7 is a diagram of an example robotic surgical system of FIG. 1.

FIG. 8 is a flowchart of an example process for steering an ultrasound catheter in a subject from a current orientation to a target orientation for a quantification task associated with an anatomical structure of interest of the subject.

FIG. 9 is a diagram of steering guidance information for a set of steering components of an ultrasound catheter.

FIG. 10 is a diagram of steering guidance information for a set of steering components of an ultrasound catheter relative to a display of the ultrasound catheter.

FIG. 11 is a diagram of steering guidance information for an ultrasound catheter.

FIG. 12 is a diagram of steering guidance information for an ultrasound catheter.

### DETAILED DESCRIPTION

As addressed above, the orientation of an ultrasound catheter relative to an anatomical structure of interest may affect the quality, or accuracy, of a quantification task associated with the anatomical structure of interest. Further, as addressed above, an ultrasound catheter may be steered using a set of steering components. A user of an ultrasound catheter might find it difficult, non-intuitive, and/or error-prone to orient the transducer of the ultrasound catheter relative to the anatomical structure of interest in a manner that allows for accurate performance of the quantification task.

Some embodiments herein provide a steering system that is configured to receive a selection of an anatomical structure of interest of a subject and a quantification task associated with the anatomical structure of interest of the subject; determine a current orientation of an ultrasound catheter in the subject; determine a target orientation of the ultrasound catheter in the subject for the quantification task associated with the anatomical structure of interest of the subject; determine steering information for steering the ultrasound catheter in the subject from the current orientation to the target orientation; and perform an action based on the steering information.

In this way, some embodiments herein provide for a steering system that more accurately and efficiently orients an ultrasound catheter relative to an anatomical structure of interest of a subject for a quantification task associated with the anatomical structure of interest. Further, in this way, some embodiments herein provide for more accurate results associated with quantification tasks, which thereby improves the efficacy of the quantification tasks and/or procedures involving the quantification tasks. Accordingly, some embodiments herein provide an improvement in the technical field of catheter-based echocardiography, provide a technical improvement to systems associated with catheter-based echocardiography, and provide an improvement to interventional procedures involving catheter-based echocardiography.

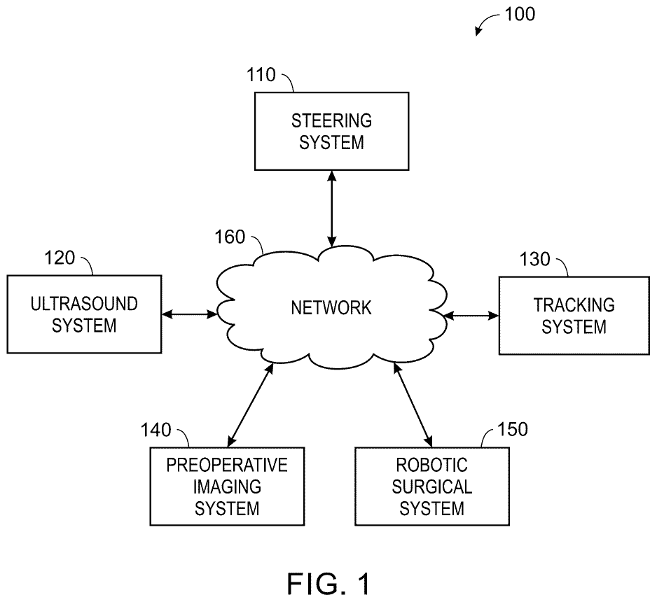

FIG. 1 is a diagram of an example system 100 for steering an ultrasound catheter in a subject from a current orientation to a target orientation for a quantification task associated with an anatomical structure of interest of the subject. As shown in FIG. 1, the system 100 may include a steering system 110, an ultrasound system 120, a tracking system 130, a preoperative imaging system 140, a robotic surgical system 150, and a network 160.

The steering system 110 may be configured to receive a selection of an anatomical structure of interest of a subject and a quantification task associated with the anatomical structure of interest of the subject; determine a current orientation of an ultrasound catheter in the subject; determine a target orientation of the ultrasound catheter in the subject for the quantification task associated with the anatomical structure of interest of the subject; determine steering information for steering the ultrasound catheter in the subject from the current orientation to the target orientation; and perform an action based on the steering information. For example, the steering system 110 may be a standalone computing system, a server, an ultrasound system, a medical imaging system, a robotic surgical system, or the like.

The ultrasound system 120 may be configured to acquire ultrasound data of an anatomical structure of interest of a subject, and perform a quantification task based on the ultrasound data. Additionally, or alternatively, the ultrasound system 120 may be configured to generate an ultrasound image of the anatomical structure of interest of the subject based on the ultrasound data. For example, the ultrasound system 120 may be a two-dimensional (2D) ultrasound system, a 3D ultrasound system, a four-dimensional (4D) ultrasound system, a Doppler ultrasound system, or the like.

The tracking system 130 may be configured to acquire tracking data corresponding to a tracked instrument. For example, the tracking system 130 may be an electromagnetic tracking system, an optical tracking system, an acoustic tracking system, an inertial tracking system, or the like. The tracked instrument may be an ultrasound catheter, an interventional device (e.g., a catheter, a needle, or the like), or the like.

The preoperative imaging system 140 may be configured to acquire preoperative imaging data. For example, the preoperative imaging system 140 may be a computed tomography (CT) system, a magnetic resonance imaging (MRI) system, an ultrasound system, an X-ray system, a positron emission tomography (PET) device, or the like

The robotic surgical system 150 may be configured to perform an interventional procedure. For example, the robotic surgical system 150 may be an autonomous system, a manual system that is controlled by a clinician, a semi-autonomous system, or the like.

The network 160 may be configured to permit communication between the steering system 110, the ultrasound system 120, the tracking system 130, the preoperative imaging system 140, and the robotic surgical system 150. For example, the network 160 may be a local area network (LAN), a wide area network (WAN), a metropolitan area network (MAN), a cellular network, a private network, an ad hoc network, an intranet, the Internet, a fiber optic-based network, or the like, and/or a combination of these or other types of networks.

The subject may be a patient, an animal, a phantom, an object, or the like. The anatomical structure of interest of the subject may be any anatomical structure of the subject. For example, the anatomical structure of interest may be the heart, the lungs, the brain, the liver, the pancreas, or the like.

The number and arrangement of the systems of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

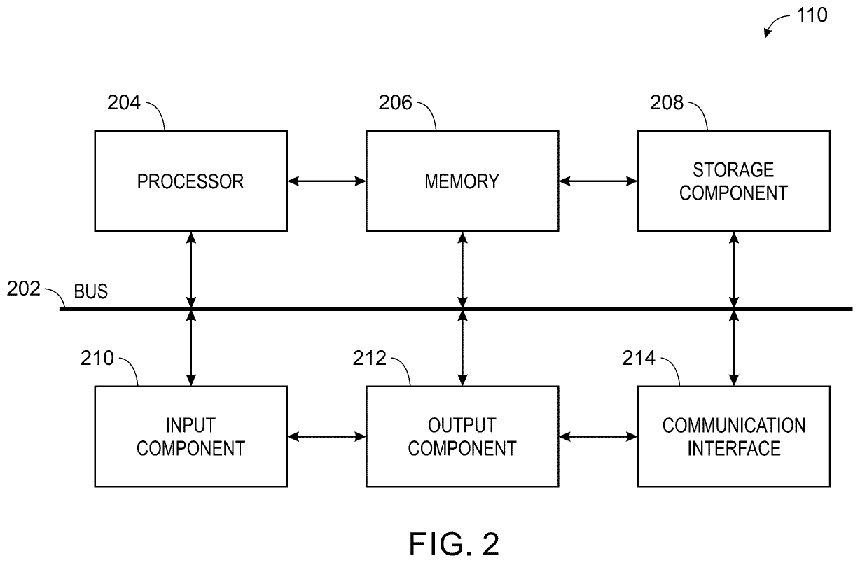

FIG. 2 is a diagram of an example steering system 110 of FIG. 1. As shown in FIG. 2, the steering system 110 may include a bus 202, a processor 204, a memory 206, a storage component 208, an input component 210, an output component 212, and a communication interface 214.

The bus 202 includes a component that permits communication among the components of the steering system 110. The processor 204 may be implemented in hardware, firmware, or a combination of hardware and software. The processor 204 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 204 may include one or more processors capable of being programmed to perform a function. The processor 204 may include one or more processors 204 configured to perform the operations described herein. For example, a single processor 204 may be configured to perform all of the operations described herein. Alternatively, multiple processors 204, collectively, may be configured to perform all of the operations described herein, and each of the multiple processors 204 may be configured to perform a subset of the operations descried herein. For example, a first processor 204 may perform a first subset of the operations described herein, a second processor 204 may be configured to perform a second subset of the operations described herein, etc.

The memory 206 may include a random access memory (RAM), a read only memory (ROM), and/or another type of dynamic or static storage device (e.g., a flash memory, a magnetic memory, and/or an optical memory) that stores information and/or instructions for use by the processor 204.

The storage component 208 may store information and/or software related to the operation and use of the steering system 110. For example, the storage component 208 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 210 may include a component that permits the steering system 110 to receive information, such as via user input (e.g., a touch screen display, a keyboard, a keypad, a mouse, a button, a switch, a camera, and/or a microphone). Additionally, or alternatively, the input component 210 may include a sensor for sensing information (e.g., a global positioning system (GPS) component, an accelerometer, a gyroscope, and/or an actuator). The output component 212 may include a component that provides output information from the steering system 110 (e.g., a display, a speaker for outputting sound at the output sound level, and/or one or more light-emitting diodes (LEDs)).

The communication interface 214 may include a transceiver-like component (e.g., a transceiver and/or a separate receiver and transmitter) that enables the steering system 110 to communicate with other systems, such as via a wired connection, a wireless connection, or a combination of wired and wireless connections. The communication interface 214 may permit the steering system 110 to receive information from another system and/or provide information to another system of FIG. 1. For example, the communication interface 214 may include an Ethernet interface, an optical interface, a coaxial interface, an infrared interface, a radio frequency (RF) interface, a universal serial bus (USB) interface, a Wi-Fi interface, a cellular network interface, or the like.

The steering system 110 may perform one or more processes described herein. The steering system 110 may perform these processes based on the processor 204 executing software instructions stored by a non-transitory computer-readable medium, such as the memory 206 and/or the storage component 208. A computer-readable medium may be defined herein as a non-transitory memory device. A memory device may include memory space within a single physical storage device or memory space spread across multiple physical storage devices.

The software instructions may be read into the memory 206 and/or the storage component 208 from another computer-readable medium or from another system via the communication interface 214. When executed, the software instructions stored in the memory 206 and/or the storage component 208 may cause the processor 204 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the steering system 110 may include additional components, fewer components, different components, or differently arranged components than those shown in FIG. 2. Additionally, or alternatively, a set of components (e.g., one or more components) of the steering system 110 may perform one or more functions described as being performed by another set of components of the steering system 110.

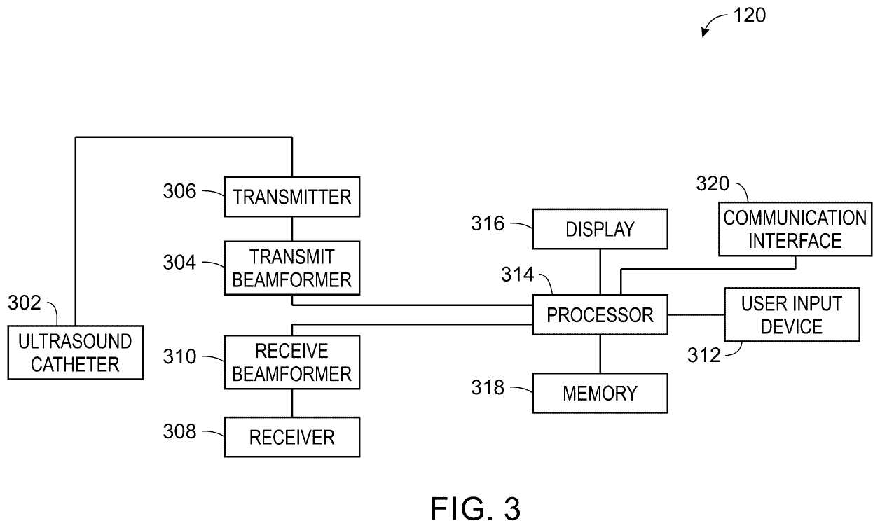

FIG. 3 is a diagram of an example ultrasound system 120 of FIG. 1. As shown in FIG. 3, the ultrasound system 120 may include an ultrasound catheter 302, a transmit beamformer 304, a transmitter 306, a receiver 308, a receive beamformer 310, a user input device 312, a processor 314, a display 316, a memory 318, and a communication interface 320. The foregoing components may be connected via wired or wireless connections.

The ultrasound catheter 302 may be configured to acquire ultrasound data. For example, the ultrasound catheter 302 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound catheter 302 may be configured to generate ultrasound signals, emit the ultrasound signals towards an anatomical structure of interest of a subject, receive echo ultrasound signals that are back-scattered from the anatomical structure of interest of the subject, generate ultrasound data based on the echo ultrasound signals, and output the ultrasound data.

According to an embodiment, the ultrasound catheter 302 may include a transducer configured to transmit ultrasound signals towards a region of interest, and receive echo signals from the region of interest, a matching layer configured to have an acoustic impedance between the region of interest to be imaged and a material of the transducer; and a damping block configured to absorb ultrasound energy.

The transmit beamformer 304 may be configured to apply delay times to electrical signals provided to the elements of the ultrasound catheter 302 to focus corresponding ultrasound signals at the anatomical structure of interest. The transmitter 306 may be configured to transmit electrical signals to the elements of the ultrasound catheter 302 to drive the elements to emit ultrasound signals towards the anatomical structure of interest. The elements of the ultrasound catheter 302 may be configured to receive the electrical signals from the transmitter 306, convert the electrical signals into ultrasound signals, and emit the ultrasound signals towards the anatomical structure of interest. The elements of the ultrasound catheter 302 may be configured to receive echo ultrasound signals that are back-scattered by the anatomical structure of interest, convert the echo ultrasound signals into electrical signals, and provide the electrical signals to the receiver 308. The receiver 308 may be configured to receive electrical signals from the elements of the ultrasound catheter 302, and provide the electrical signals to the receive beamformer 310. The receive beamformer 310 may apply delay times to the electrical signals received from the elements of the ultrasound catheter 302.

The user input device 312 may be configured to receive a user input, and provide the user input to the processor 314. For example, the user input device 312 may be a touch screen display, a keyboard, a keypad, a mouse, a button, a switch, a microphone, or the like. Additionally, or alternatively, the user input device 312 may be configured to sense information. For example, the user input device 312 may sense information from an electro-magnetic positioning system, an inertial measurement system, an accelerometer, a gyroscope, an actuator, or the like.

The processor 314 may be configured to perform the operations as described herein. For example, the processor 314 may be a CPU, a GPU, an APU, a microprocessor, a microcontroller, a DSP, an FPGA, an ASIC, or the like. The processor 314 may be implemented in hardware, firmware, or a combination of hardware and software. The processor 314 may include one or more processors 314 configured to perform the operations described herein. For example, a single processor 314 may be configured to perform all of the operations described herein. Alternatively, multiple processors 314, collectively, may be configured to perform all of the operations described herein, and each of the multiple processors 314 may be configured to perform a subset of the operations described herein. For example, a first processor 314 may perform a first subset of the operations described herein, a second processor 314 may be configured to perform a second subset of the operations described herein, etc.

The processor 314 may be configured to control the ultrasound catheter 302 to acquire ultrasound data. The processor 314 may be configured to control which of the elements of the ultrasound catheter 302 are active, and control the shape of a beam emitted from the ultrasound catheter 302. The processor 314 may generate ultrasound images for display. For example, the processor 314 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 316 may be configured to display information. For example, the display 316 may be a monitor, an LED display, a cathode ray tube, a projector display, a touchscreen, tablet computer, mobile phone, or the like. The display 316 may display ultrasound images based on the ultrasound data in real-time. For example, the display 316 may display the ultrasound images within one second, two seconds, five seconds, etc., of the ultrasound data being acquired by the ultrasound catheter 310.

The memory 318 may be configured to store information and/or instructions for use by the processor 314. The memory 318 may be a non-transitory computer-readable medium. For example, the memory 318 may be a RAM, a ROM, a flash memory, a magnetic memory, an optical memory, or the like. The memory 318 may be configured to store instructions that, when executed by the processor 314, cause the processor 314 to perform the operations described herein.

The communication interface 320 may be configured to enable the processor 314 to communicate with other systems, such as via a wired connection, a wireless connection, or a combination of wired and wireless connections. For example, the communication interface 320 may include an Ethernet interface, an optical interface, a coaxial interface, an infrared interface, an RF interface, a USB interface, a Wi-Fi interface, a cellular network interface, or the like.

The number and arrangement of the components of the ultrasound system 120 shown in FIG. 3 are provided as an example. In practice, the ultrasound system 120 may include additional components, fewer components, different components, or differently arranged components than those shown in FIG. 3. Additionally, or alternatively, a set of components (e.g., one or more components) of the ultrasound system 120 may perform one or more functions described as being performed by another set of components of the ultrasound system 120.

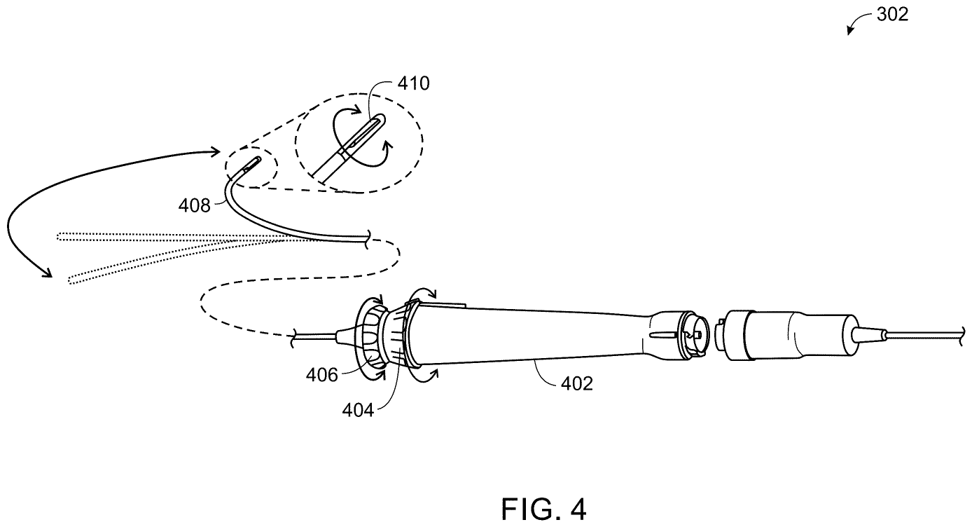

FIG. 4 is a diagram of an example ultrasound catheter 302 of the ultrasound system 120 of FIG. 3. As shown in FIG. 4, the ultrasound catheter 302 may include a handle 402, a first steering component 404, a second steering component 406, a catheter portion 408, and a transducer 410.

The handle 402 may be provided at a proximal end of the ultrasound catheter 302, and may be manipulated by a user during usage of the ultrasound catheter 302. The handle 402 may connect to a console of the ultrasound system 120 via a cable.

The first steering component 404 may be configured to steer the transducer 410 of the ultrasound catheter 302. For example, the first steering component may be configured to rotate the transducer 410 about a first axis. The first axis may be a pitch axis, a yaw axis, or the like.

The second steering component 406 may be configured to steer the transducer 410 of the ultrasound catheter 302. For example, the second steering component may be configured to rotate the transducer 410 about a second axis. The second axis may be a roll axis.

As shown in FIG. 4, the first steering component 404 and the second steering component 406 may be rotational knobs. However, it should be understood that the first steering component 404 and/or the and the second steering component 406 may be another type of steering component in other embodiments, such as a button, a lever, a slider, or the like. Further, although FIG. 4 depicts two steering components, it should be understood that the ultrasound catheter 302 may include any number of steering components that steer the transducer 410.

The catheter portion 408 may include a proximal end located adjacent to the handle 402, and include a distal end. The transducer 410 may be located at, or adjacent to, the distal end of the catheter portion 408. The catheter portion 408 may be configured to be navigated through the body of the subject towards, and to, the anatomical structure of interest.

The transducer 410 may be configured to acquire ultrasound data of the anatomical structure of interest. For example, the transducer 410 may emit ultrasound signals towards the anatomical structure of interest, and may receive echo signals reflected by, or back-scattered from, the anatomical structure of interest.

The number and arrangement of the components of the ultrasound catheter 302 shown in FIG. 4 are provided as an example. In practice, the ultrasound catheter 302 may include additional components, fewer components, different components, or differently arranged components than those shown in FIG. 4. Additionally, or alternatively, a set of components (e.g., one or more components) of the ultrasound catheter 302 may perform one or more functions described as being performed by another set of components of the ultrasound catheter 302.

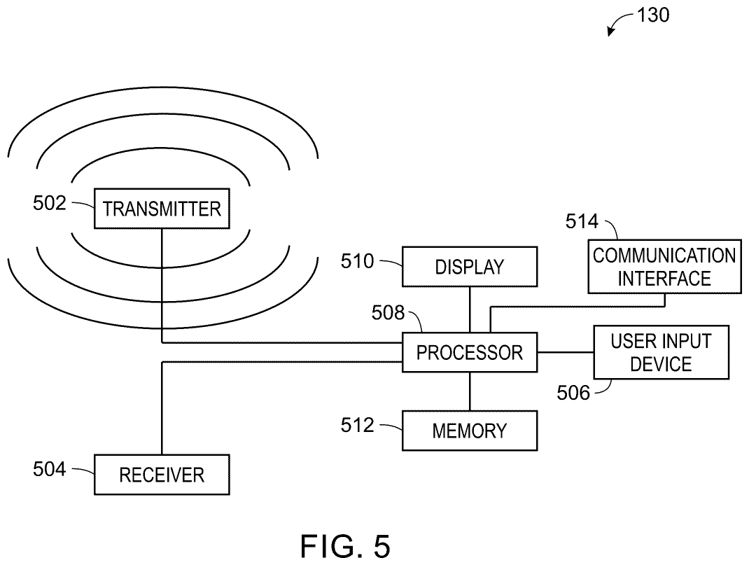

FIG. 5 is a diagram of an example tracking system 130 of FIG. 1. As shown in FIG. 4, the tracking system 130 may be an electromagnetic tracking system, and may include a transmitter 502, a receiver 504, a user input device 506, a processor 508, a display 510, a memory 512, and a communication interface 514.

The transmitter 502 may be configured to generate a magnetic field. The receiver 504 may be configured to output a signal in response to the magnetic field generated by the transmitter 502. The processor 508 may receive the output signal from the receiver 504, and acquire tracking data that identifies a position and/or an orientation of the receiver 504. The receiver 504 may be attached to, integrated with, provided in, etc., a tracked instrument. For example, according to an embodiment, the receiver 504 may be attached to the ultrasound catheter 302 to track a position and/or an orientation of the ultrasound catheter 302. Alternatively, the receiver 504 may be attached to an interventional device to track a position and/or an orientation of the interventional device. The interventional device may be a catheter, a needle, or the like.

The user input device 506 may be configured to receive a user input, and provide the user input to the processor 508. For example, the user input device 506 may be a touch screen display, a keyboard, a keypad, a mouse, a button, a switch, a microphone, or the like. Additionally, or alternatively, the user input device 506 may be configured to sense information. For example, the user input device 506 may sense information from an electro-magnetic positioning system, an inertial measurement system, an accelerometer, a gyroscope, an actuator, or the like.

The processor 508 may be configured to perform the operations as described herein. For example, the processor 508 may be a CPU, a GPU, an APU, a microprocessor, a microcontroller, a DSP, an FPGA, an ASIC, or the like. The processor 508 may be implemented in hardware, firmware, or a combination of hardware and software. The processor 508 may include one or more processors 508 configured to perform the operations described herein. For example, a single processor 508 may be configured to perform all of the operations described herein. Alternatively, multiple processors 508, collectively, may be configured to perform all of the operations described herein, and each of the multiple processors 508 may be configured to perform a subset of the operations described herein. For example, a first processor 508 may perform a first subset of the operations described herein, a second processor 508 may be configured to perform a second subset of the operations described herein, etc.

The processor 508 may be configured to control the transmitter 502 to acquire tracking data. The processor 508 may be configured to control excitations of the transmitter 502 to generate a magnetic field. The processor 508 may acquire tracking data based on controlling the transmitter 502.

The display 510 may be configured to display information. For example, the display 510 may be a monitor, an LED display, a cathode ray tube, a projector display, a touchscreen, tablet computer, mobile phone, or the like. The display 510 may display the tracking data in real-time. For example, the display 510 may display the tracking data within one second, two seconds, five seconds, etc., of the tracking data being acquired.

The memory 512 may be configured to store information and/or instructions for use by the processor 508. The memory 512 may be a non-transitory computer-readable medium. For example, the memory 512 may be a RAM, a ROM, a flash memory, a magnetic memory, an optical memory, or the like. The memory 512 may be configured to store instructions that, when executed by the processor 508, cause the processor 508 to perform the operations described herein.

The communication interface 514 may be configured to enable the processor 508 to communicate with other systems, such as via a wired connection, a wireless connection, or a combination of wired and wireless connections. For example, the communication interface 514 may include an Ethernet interface, an optical interface, a coaxial interface, an infrared interface, an RF interface, a USB interface, a Wi-Fi interface, a cellular network interface, or the like.

The number and arrangement of the components of the tracking system 130 shown in FIG. 5 are provided as an example. In practice, the tracking system 130 may include additional components, fewer components, different components, or differently arranged components than those shown in FIG. 5. Additionally, or alternatively, a set of components (e.g., one or more components) of the tracking system 130 may perform one or more functions described as being performed by another set of components of the tracking system 130.

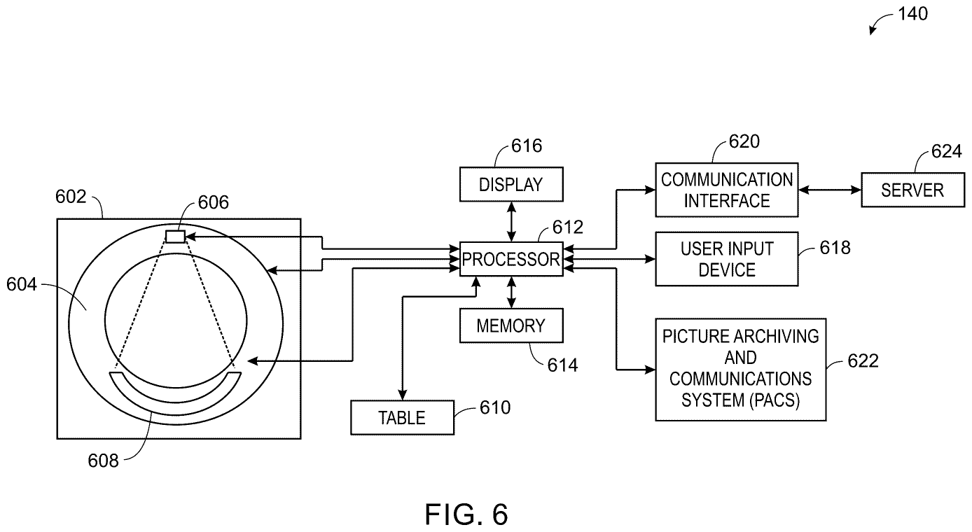

FIG. 6 is a diagram of an example preoperative imaging system 140 of FIG. 1. As shown in FIG. 4, the preoperative imaging system 140 may include a gantry 602, a rotational frame 604, an X-ray source 606, an X-ray detector 608, a table 610, a processor 612, a memory 614, a display 616, a user input device 618, a communication interface 620, a picture archiving and communications system (PACS) 622, and a server 624.

The processor 612 may be configured to control operations of the preoperative imaging system 140. For example, the processor 612 may be a CPU, a GPU, an APU, a microprocessor, a microcontroller, a DSP, an FPGA, an ASIC, or the like. The processor 612 may be implemented in hardware, firmware, or a combination of hardware and software. The processor 612 may include one or more processors 612 configured to perform the operations described herein. For example, a single processor 612 may be configured to perform all of the operations described herein. Alternatively, multiple processors 612, collectively, may be configured to perform all of the operations described herein, and each of the multiple processors 612 may be configured to perform a subset of the operations descried herein. For example, a first processor 612 may perform a first subset of the operations described herein, a second processor 612 may be configured to perform a second subset of the operations described herein, etc.

The processor 612 may be configured to control the gantry 602, movement of the rotational frame 604, the X-ray source 606, the X-ray detector 608, and movement of the table 610.

The memory 614 may be configured to store information and/or instructions for use by the processor 612. The memory 614 may be a non-transitory computer-readable medium. For example, the memory 614 may be a RAM, a ROM, a flash memory, a magnetic memory, an optical memory, or the like. The memory 614 may be configured to store instructions that, when executed by the processor 612, cause the processor 612 to perform the operations described herein.

The display 616 may be configured to display information. For example, the display 616 may be a monitor, an LED display, a cathode ray tube, a projector display, a touchscreen, tablet computer, mobile phone, or the like.

The user input device 618 may be configured to receive a user input, and provide the user input to the processor 612. For example, the user input device 618 may be a touch screen display, a keyboard, a keypad, a mouse, a button, a switch, a microphone, or the like. Additionally, or alternatively, the user input device 618 may be configured to sense information. For example, the user input device 618 may sense information from an electro-magnetic positioning system, an inertial measurement system, an accelerometer, a gyroscope, an actuator, or the like.

The communication interface 620 may be configured to enable the processor 612 to communicate with other devices, such as via a wired connection, a wireless connection, or a combination of wired and wireless connections. For example, the communication interface 620 may include an Ethernet interface, an optical interface, a coaxial interface, an infrared interface, an RF interface, a USB interface, a Wi-Fi interface, a cellular network interface, or the like. The PACS 422 may be configured to communicate with external systems and/or networks to permit users at various locations to access the medical image. The server 624 may be configured to store one or more models as described herein. For example, the server 624 may be an on-premises server, a cloud server, a virtual machine, or the like.

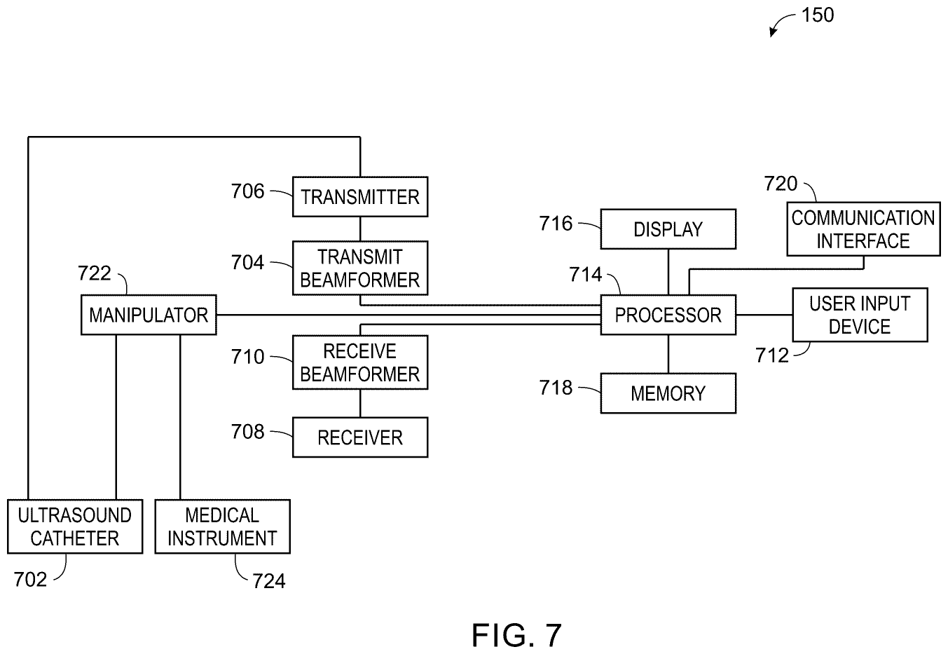

FIG. 7 is a diagram of an example robotic surgical system 150 of FIG. 1. As shown in FIG. 7, the robotic surgical system 150 may include an ultrasound catheter 702, a transmit beamformer 704, a transmitter 706, a receiver 708, a receive beamformer 710, a user input device 712, a processor 714, a display 716, a memory 718, a communication interface 720, a manipulator 722, and a medical instrument 724. The foregoing components may be connected via wired or wireless connections.

The ultrasound catheter 702, the transmit beamformer 704, the transmitter 706, the receiver 708, the receive beamformer 710, the user input device 712, the processor 714, the display 716, the memory 718, and/or communication interface 720 may be substantially similar, or similar to, the ultrasound catheter 302, the transmit beamformer 304, the transmitter 306, the receiver 308, the receive beamformer 310, the user input device 312, the processor 314, the display 316, the memory 318, and/or the communication interface 320, respectively, as described above in connection with FIG. 3.

The manipulator 722 may be configured to manipulate the ultrasound catheter 702 and/or the medical instrument 724. For example, the manipulator 722 may include a motor, an actuator, an end-effector, an arm, and/or the like.

The medical instrument 724 may be any device that can be navigated through a region of interest of a subject. For example, the medical instrument 724 may be a catheter, a needle, a guidewire, a trocar, a cannula, or the like. The medical instrument 724 may be used for various interventional procedures involving the region of interest. For example, a catheter may be used for delivering a stent to an occluded blood vessel, inserting a mitral valve clip, closing a left atrial appendage, ablating tissue, analyzing cardiac function, removing a thrombus from an occluded blood vessel, or the like. Alternatively, the medical instrument 724 may be an implantable device that is to be implanted in the subject. For example, the medical instrument 724 may be a pacemaker, a stent, a defibrillator, a left ventricular assist device, a valve clip, or the like. Alternatively, the medical instrument 724 may be any object that can be navigated throughout the region of interest of the subject.

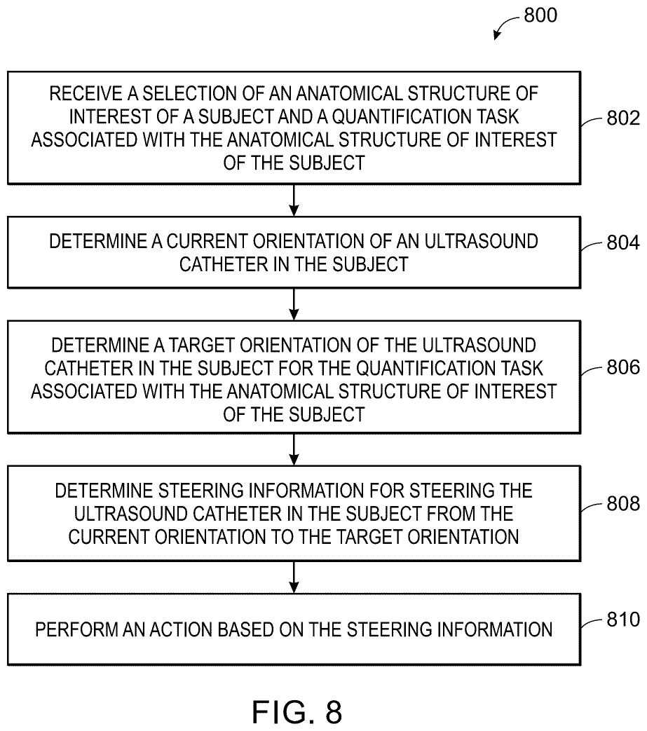

FIG. 8 is a flowchart of an example process 800 for steering an ultrasound catheter in a subject from a current orientation to a target orientation for a quantification task associated with an anatomical structure of interest of the subject. According to an embodiment, the steering system 110 may be configured to perform one or more operations of the process 800. However, it should be understood that, in other embodiments, one or more other systems may be configured to perform one or more operations of the process 800.

As shown in FIG. 8, the process 800 may include receiving a selection of an anatomical structure of interest of a subject and a quantification task associated with the anatomical structure of interest of the subject (operation 802). For example, the steering system 110 may receive a selection of an anatomical structure of interest of a subject and a quantification task associated with the anatomical structure of interest of the subject.

The anatomical structure of interest may be any anatomical structure of the subject. For example, the anatomical structure of interest may be the heart, a lung, the liver, the pancreas, or the like. As further examples, the anatomical structure of interest may be the aorta, a pulmonary artery, the tricuspid valve, the mitral valve, or the like.

According to an embodiment, the quantification task may be the measurement of a blood flow parameter associated with the anatomical structure of interest. For example, the blood flow parameter may be a blood flow direction, a blood flow velocity, a blood flow rate, or the like. Additionally, or alternatively, the quantification task may be the measurement of a dimension of the anatomical structure of interest. Additionally, or alternatively, the quantification task may be a set of measurements for a set of anatomical structures of interest according to a protocol. For example, the quantification task may include a set of measurements of blood flow parameters of a set of anatomical structures of interest. For instance, a cardiologist may be interested in blood flow parameters of major arteries, veins, or valves (e.g., the aorta, the pulmonary artery, the mitral valve, the tricuspid valve, or any other set of structures that can be specified by the cardiologist).

According to an embodiment, the steering system 110 may receive the selection based on a user input. For example, the user may input, or select, information identifying the anatomical structure of interest and the quantification task associated with the anatomical structure of interest. As an example, the user may select the anatomical structure of interest from a displayed list of anatomical structures of interest displayed on a user interface, and/or may select the quantification task from a displayed list of quantification tasks displayed on the user interface. Alternatively, the steering system 110 may receive the selection based on predetermined information. For example, the steering system 110 may receive the selection based on predetermined information stored by the steering system 110. Alternatively, the steering system 110 may receive the selection based on a procedure associated with the ultrasound catheter 302. For example, the steering system 110 may receive information identifying a procedure to be performed using the ultrasound catheter 302, and receive the selection based on the procedure. Alternatively, the steering system 110 may receive the selection based on a particular anatomical structure of interest that is being imaged by the ultrasound catheter 302. For example, the user may manipulate the ultrasound catheter 302 to image a particular anatomical structure of interest, and the steering system 110 may receive the selection based on the particular anatomical structure of interest being imaged.

As further shown in FIG. 8, the process 800 may include determining a current orientation of an ultrasound catheter in the subject (operation 804). For example, the steering system 110 may determine a current orientation of the ultrasound catheter 302 in the subject.

The user may navigate the ultrasound catheter 302 to a region of interest of the subject, and may manipulate the ultrasound catheter 302 to orient the ultrasound catheter 302 relative to the anatomical structure of interest. For example, the user may manipulate the first steering component 404 and/or the second steering component 406 of the ultrasound catheter 302 to orient the transducer 410 relative to the anatomical structure of interest.

According to an embodiment, the steering system 110 may determine a current orientation of the ultrasound catheter 302 based on tracking data acquired by the tracking system 130. For example, the steering system 110 may receive tracking data of the ultrasound catheter 302 acquired by the tracking system 130, and determine the current orientation of the ultrasound catheter 302 based on the tracking data.

According to an embodiment, the steering system 110 may determine a current orientation of the ultrasound catheter 302 based on ultrasound data acquired by the ultrasound catheter 302. For example, the steering system 110 may receive ultrasound data acquired by the ultrasound catheter 302, and determine the current orientation of the ultrasound catheter 302 based on the ultrasound data. According to an embodiment, the steering system 110 may determine the current orientation of the ultrasound catheter 302 using the ultrasound data and an image processing technique. For example, the image processing technique may be a segmentation technique, a pattern matching technique, a feature extraction technique, an image analysis technique, an edge detection technique, an image registration technique, or the like. In this case, the steering system 110 may analyze the ultrasound data using the image processing technique, determine one or more anatomical structures in the ultrasound data, and determine the current orientation of the ultrasound catheter 302 based on the one or more anatomical structures in the ultrasound data.

According to an embodiment, the steering system 110 may may determine a current orientation of the ultrasound catheter 302 using ultrasound data and an AI model. For example, the steering system 110 may input the ultrasound data into the AI model, and determine a current orientation of the ultrasound catheter 302 based on an output of the AI model. The AI model may be a convolutional neural network (CNN) model, a residual neural network, a random forest model, a decision tree model, an artificial neural network (ANN), a Naïve Bayes model, a decision tree, a recurrent neural network (RNN), a logistic regression model, a support vector machine, or the like. In this case, the AI model may be trained to receive ultrasound data, analyze the ultrasound data to determine a current orientation of the ultrasound catheter 302, and output information that identifies the current orientation of the ultrasound catheter 302. The training data may include known ultrasound data that is correlated with known orientations of the ultrasound catheter 302.

According to an embodiment, the steering system 110 may determine a current orientation of the ultrasound catheter 302 using preoperative imaging data of the region of interest acquired by the preoperative imaging system 140. For example, the steering system 110 may register the ultrasound data with preoperative imaging data, of a preoperative imaging dataset, acquired by the preoperative imaging system 140 that identifies the anatomical structure of interest, and determine the current orientation of the ultrasound catheter 302 based on registering the ultrasound data with the preoperative imaging data.

According to an embodiment, the steering system 110 may determine a current orientation of the ultrasound catheter 302 using a model of a region of interest that includes the anatomical structure of interest. For example, the steering system 110 may determine a current view of the ultrasound catheter 302 based on the ultrasound data and a view recognition technique, and determine a corresponding cut plane of the model based on the current view of the ultrasound catheter 302. The steering system 110 may determine a position in the model space of the model that generates the cut plane, and determine a current orientation of the ultrasound catheter 302 based on the position in the model space.

As further shown in FIG. 8, the process 800 may include determining a target orientation of the ultrasound catheter in the subject for the quantification task associated with the anatomical structure of interest of the subject (operation 806). For example, the steering system 110 may determine a target orientation of the ultrasound catheter 302 in the subject for the quantification task associated with the anatomical structure of interest of the subject.

The target orientation of the ultrasound catheter 302 may be an orientation of the ultrasound catheter 302 that permits the quantification task to be performed. In some embodiments, the target orientation may be an orientation that permits the quantification task to be performed most accurately. Alternatively, the target orientation may be an orientation that permits the quantification task to be performed more accurately than as compared to the current orientation of the ultrasound catheter 302.

According to an embodiment, the steering system 110 may determine the target orientation based on the quantification task to be performed. For example, if the quantification task is the measurement of a blood flow parameter associated with the anatomical structure of interest, then the target orientation may be an orientation of the ultrasound catheter 302 in which an insonation angle between an ultrasound beam and blood flow is less than a threshold, is minimized, is less than an insonation angle of the current orientation of the ultrasound catheter 302, or the like. As another example, if the quantification task is measurement of a dimension of the anatomical structure of interest, then the target orientation may be an orientation of the ultrasound catheter 302 in which an ultrasound image of the anatomical structure of interest depicts a largest dimension of the anatomical structure of interest, in which an ultrasound image is orthogonal to a longitudinal axis of the anatomical structure of interest, or the like.

According to an embodiment, the steering system 110 may determine the target orientation based on ultrasound data acquired by the ultrasound catheter 302. For example, the steering system 110 may analyze the ultrasound data using an image processing technique, and determine the target orientation based on performing the image processing technique. As an example, if the anatomical structure of interest is a blood vessel and the quantification task is a measurement of a blood flow parameter of the blood vessel, then the steering system 110 may determine a target orientation that orients the ultrasound beam of the ultrasound catheter 302 to be parallel, or substantially parallel, with the longitudinal axis of the blood vessel as determined based on image processing technique. According to an embodiment, the steering system 110 may determine the target orientation based on an insonation angle between an ultrasound beam of the ultrasound catheter 302 and blood flow of the anatomical structure of interest. For example, the steering system 110 may determine an insonation angle, and determine the target orientation based on the insonation angle, such that the insonation angle of the target orientation is minimized or is, at least, less than an insonation angle of the current orientation.

As another example, if the anatomical structure of interest is a blood vessel and the quantification task is a measurement of a dimension of the blood vessel, then the steering system 110 may determine a target orientation that results in an ultrasound image that includes a largest diameter of the blood vessel and that is orthogonal to a longitudinal axis of the blood vessel.

According to an embodiment, the steering system 110 may determine the target orientation based on ultrasound data acquired by the ultrasound catheter 302. For example, the steering system 110 may analyze the ultrasound data using a flow imaging technique, and determine the target orientation based on performing the flow imaging technique. The flow imaging technique may be power Doppler imaging, color velocity imaging, vector flow imaging, spectral Doppler imaging, or the like. As an example, if the anatomical structure of interest is a blood vessel and the quantification task is a measurement of a blood flow parameter of the blood vessel, then the steering system 110 may determine a target orientation that orients the ultrasound beam of the ultrasound catheter 302 to be parallel, or substantially parallel, with the longitudinal axis of the blood vessel as determined based on the flow imaging technique.

According to an embodiment, the steering system 110 may determine the target orientation based on an AI model. For example, the steering system 110 may input the ultrasound data, the current orientation, information identifying the quantification task, information identifying the anatomical structure of interest, and/or the like, into the AI model, and determine the target orientation of the ultrasound catheter 302 based on an output of the AI model.

According to an embodiment, the steering system 110 may determine the target orientation based on preoperative imaging data acquired by the preoperative imaging system 140. For example, the steering system 110 may analyze the preoperative imaging data of the anatomical structure of interest, and determine the target orientation based on analyzing the preoperative imaging data.

According to an embodiment, the steering system 110 may determine the target orientation based on the model of the region of interest. For example, the steering system 110 may analyze the model of the region of interest, and determine the target orientation based on analyzing the model of the region of interest. According to an embodiment, the steering system 110 may determine a target plane using the model.

As further shown in FIG. 8, the process 800 may include determining steering information for steering the ultrasound catheter in the subject from the current orientation to the target orientation (operation 808). For example, the steering system 110 may determine steering information for steering the ultrasound catheter in the subject from the current orientation to the target orientation.

The steering information may be information that identifies motion parameters for steering the ultrasound catheter 302 from the current orientation to the target orientation. The motion parameters may be a rotation about a yaw axis, a rotation about a pitch axis, a rotation about a roll axis, a translation along a pitch axis, or the like.

According to an embodiment, the steering system 110 may determine the steering information based on the current orientation and the target orientation. For example, the steering system 110 may determine current orientation coordinates of the ultrasound catheter 302 and target orientation coordinates of the ultrasound catheter 302 in a coordinate space, and determine the steering information based on the current orientation coordinates and the target orientation coordinates. The coordinate space may be a coordinate space of a catheterization laboratory, the steering system 110, the ultrasound system 120, the tracking system 130, the preoperative imaging system 140, the robotic surgical system 150, the model of the region of interest, or the like.

According to an embodiment, the steering system 110 may determine the steering information based on the cut plane and the target plane, as described above. For example, the steering system 110 may determine a transformation map between the cut plane and the target plane. As a particular example, the steering system may determine a normal vector for the cut plane and a normal vector of the target plane, and determine axis rotation that is perpendicular to both of the normal vectors. Further, the steering system 110 may determine an angle between the normal vectors using dot product. Further, the steering system 110 may generate a 3×3 rotation matrix using the axis-angle representation. Further still, the steering system 110 may determine the translation by rotating one of the planes, locating a point on the plane, projecting the point to the next plane, and determining the distance. Further, the steering system 110 may combine the rotation and the translation to determine an overall transformation map. For example, the transformation map may be presented as:

$${transformation}_{mao} = [ \text{} \begin{matrix}{R}_{3 \times 3} & {T}_{3 \times 1} \\ {0}_{1 \times 3} & 1\end{matrix} \text{} ]$$

According to an embodiment, the steering information may identify particular amounts of movement of each of the respective steering components of the ultrasound catheter 302. For example, the steering information may identify amounts of movement of one or more steering components of the ultrasound catheter 302 that, when effectuated, steer the ultrasound catheter 302 from the current orientation to the target orientation.

As further shown in FIG. 8, the process 800 may include performing an action based on the steering information (operation 810). For example, the steering system 110 may perform an action based on the steering information.

According to an embodiment, the action may be displaying steering guidance information based on the steering information. The steering guidance information may guide the user to steer the ultrasound catheter 302 from the current orientation to the target orientation. For example, the steering guidance information may be one or more visual indicators that respectively identify amounts of movement of the one or more steering components of the ultrasound catheter 302. Additionally, or alternatively, the steering guidance information may be a visual indicator corresponding to the current orientation of the ultrasound catheter 302 and a visual indicator corresponds to the target orientation of the ultrasound catheter 302.

Additionally, or alternatively, the steering guidance information may be a current quantification plane of the ultrasound catheter 302 and a target quantification plane of the ultrasound catheter 302. The current quantification plane may be a plane in which the ultrasound catheter 302 is currently acquiring ultrasound data for, and the target quantification plane may be a plane in which the ultrasound catheter 302 will acquire ultrasound data for after the ultrasound catheter 302 is steered to the target orientation. Additionally, or alternatively, the steering guidance information may be one or more visual indicators that are displayed along a representation of the ultrasound catheter 302 and that instruct the user to manipulate the one or more steering components. Additionally, or alternatively, the steering guidance information may be one or more visual indicators that are displayed along a representation of a generic object that instructs the user to manipulate the one or more steering components. For example, the generic object may be a representation of an airplane, or the like, for which roll, pitch, and yaw are readily ascertainable.

According to an embodiment, the steering guidance information may correspond to a set of anatomical structures that are associated with the quantification task, and may guide the user to steer the ultrasound catheter 302 to a set of target positions that are respectively associated with the set of anatomical structures. For example, a protocol may involve the measurement of blood flow parameters of a set of anatomical structures. In this case, the steering guidance information may guide the user to steer the ultrasound catheter 302 to a first anatomical structure of the set of anatomical structures to measure a blood flow parameter of the first anatomical structure, may guide the user to steer the ultrasound catheter 302 from the first anatomical structure to a second anatomical structure, etc.

According to another embodiment, the action may be providing the steering information to the robotic surgical system 150. For example, the steering system 110 may provide the steering information to the robotic surgical system 150 to permit the robotic surgical system 150 to steer the ultrasound catheter 702. In this case, the ultrasound catheter 702 may be the underlying ultrasound catheter for which the current orientation and the target orientation were determined. In this way, the robotic surgical system 150 may use the steering guidance information to automatically steer the ultrasound catheter 702 to the target orientation.

According to another embodiment, the action may be automatically performing the quantification task based on the ultrasound catheter 302 being steered to the target orientation. For example, the steering system 110 may determine that the ultrasound catheter 302 is in the target orientation, and may automatically perform the quantification task based on the ultrasound catheter 302 being in the target orientation.

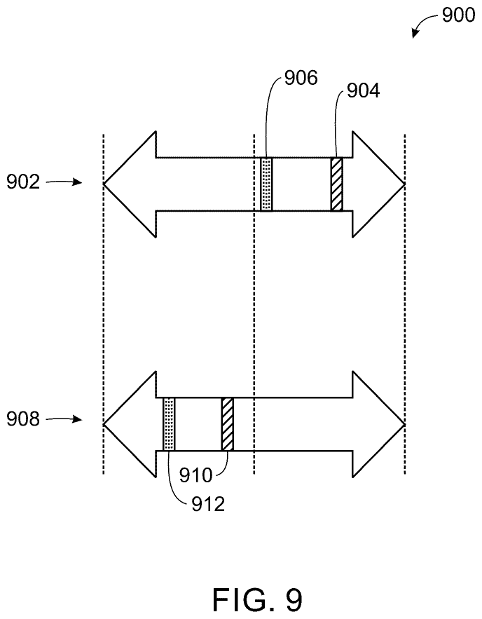

FIG. 9 is a diagram 900 of steering guidance information for a set of steering components of an ultrasound catheter. As shown in FIG. 9, the steering system 110 may display a first visual indicator 902 that corresponds to the first steering component 404 of the ultrasound catheter 302 including a current orientation indicator 904 and a target orientation indicator 906. Further, as shown in FIG. 9, the steering system 110 may display a second visual indicator 908 that corresponds to the second steering component 406 of the ultrasound catheter 302 including a current orientation indicator 910 and a target orientation indicator 912. The user may view the current orientation indicator 904 and the target orientation indicator 906 to ascertain the extent to manipulate the first steering component 404. That is, the user may manipulate the first steering component 404 which causes the current orientation indicator 904 to move. When the current orientation indicator 904 overlaps with the target orientation indicator 906, the user may determine that the first steering component 404 does not need additional manipulation. Similarly, the user may view the current orientation indicator 910 and the target orientation indicator 912 to ascertain the extent to manipulate the second steering component 406. That is, the user may manipulate the second steering component 406 which causes the current orientation indicator 910 to move. When the current orientation indicator 910 overlaps with the target orientation indicator 912, the user may determine that the second steering component 406 does not need additional manipulation.

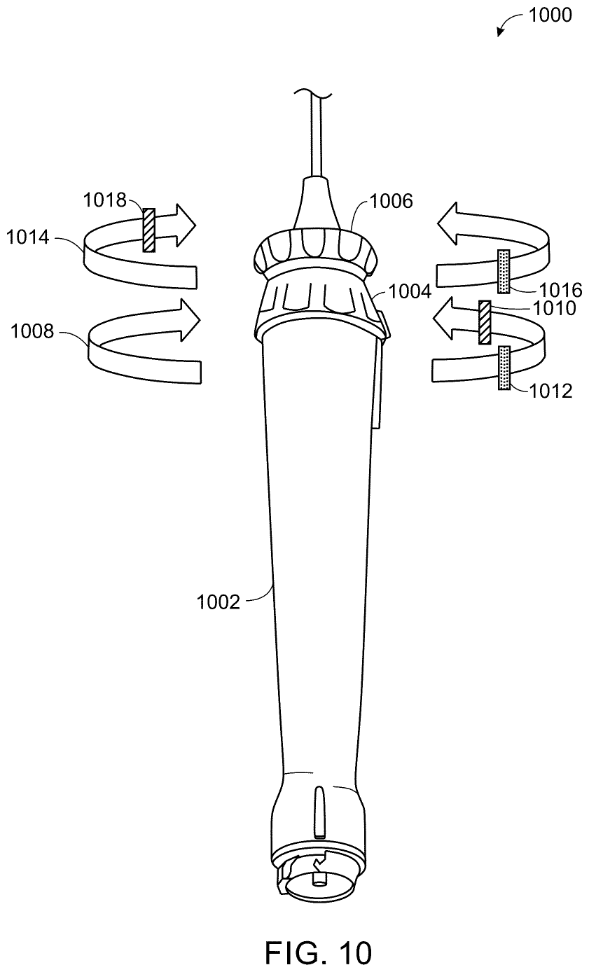

FIG. 10 is a diagram 1000 of steering guidance information for a set of steering components of an ultrasound catheter relative to a display of the ultrasound catheter. As shown in FIG. 10, the steering system 110 may display an ultrasound catheter indicator 1002 that depicts the ultrasound catheter 302, a first steering component indicator 1004 that corresponds to the first steering component 404 of the ultrasound catheter 302, and a second steering component indicator 1006 that corresponds to the second steering component 406 of the ultrasound catheter 302. Further, the steering system 110 may display a first visual indicator 1008 that corresponds to the first steering component 404 of the ultrasound catheter 302 including a current orientation indicator 1010 and a target orientation indicator 1012. Further, as shown in FIG. 10, the steering system 110 may display a second visual indicator 1014 that corresponds to the second steering component 406 of the ultrasound catheter 302 including a current orientation indicator 1016 and a target orientation indicator 1018. In this way, the first visual indicator 1008 and the second visual indicator 1014 may be displayed in relation to a visual depiction of the ultrasound catheter 302, which may improve the ability of the user to ascertain how to manipulate the first steering component 404 and the second steering component 406 to steer the ultrasound catheter 302 from the current orientation to the target orientation.

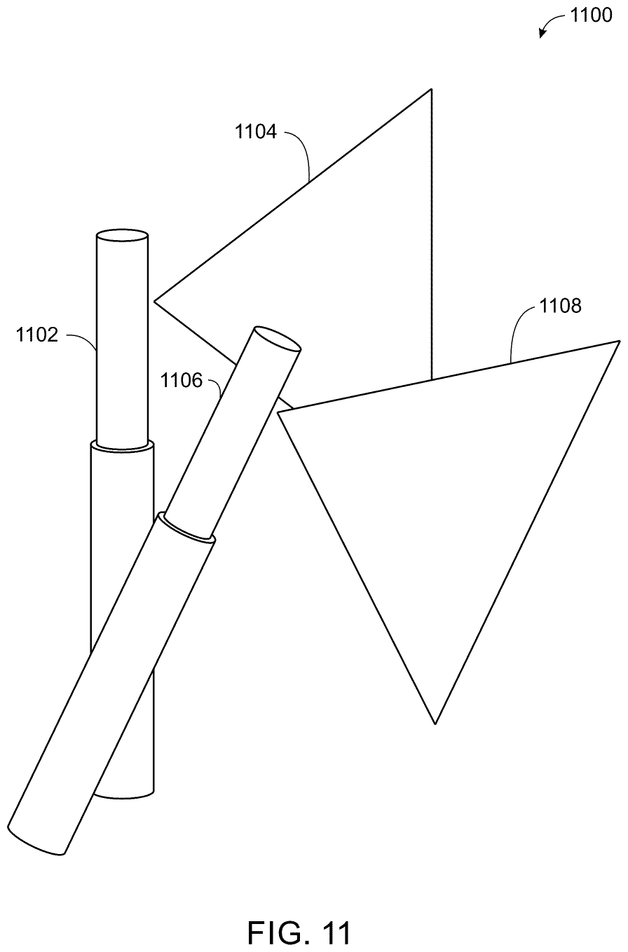

FIG. 11 is a diagram 1100 of steering guidance information for an ultrasound catheter. As shown in FIG. 11, the steering system 110 may display a visual indicator 1102 corresponding to a current orientation of the ultrasound catheter 302 and a current plane indicator 1104 corresponding to a current plane an ultrasound beam of the ultrasound catheter 302, and may display a visual indicator 1106 corresponding to a target orientation of the ultrasound catheter 302 and a target plane indicator 1108 corresponding to a target plane of the ultrasound beam of the ultrasound catheter 302. In this way, the user may manipulate the ultrasound catheter 302 which causes the visual indicator 1102 and the current plane indicator 1104 to move.

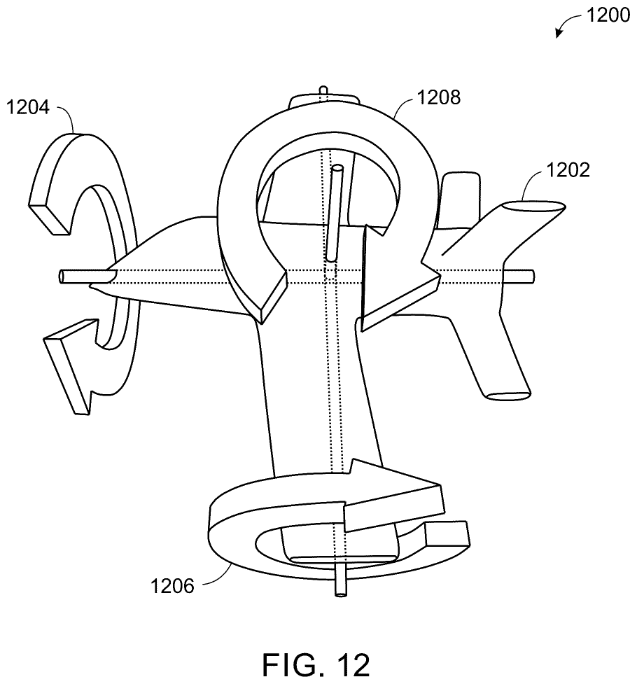

FIG. 12 is a diagram 1200 of steering guidance information for an ultrasound catheter. As shown in FIG. 12, the steering system 110 may display a visual indicator 1202 corresponding to an airplane. Further, the steering system 110 may display a visual indicator 1204 corresponding to a roll axis of the visual indicator 1202, a visual indicator 1206 corresponding to a pitch axis of the visual indicator 1202, and a visual indicator 1208 corresponding to a yaw axis of the visual indicator 1202. In this way, a user may visualize the manipulations of the ultrasound catheter 302 in reference to a more familiar object, such as the plane.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system comprising:
a memory configured to store instructions; and
one or more processors configured to execute the instructions to:
receive a selection of an anatomical structure of interest of a subject and a quantification task associated with the anatomical structure of interest of the subject;
determine a current orientation of an ultrasound catheter in the subject;
determine a target orientation of the ultrasound catheter in the subject for the quantification task associated with the anatomical structure of interest of the subject;
determine steering information for steering the ultrasound catheter in the subject from the current orientation to the target orientation; and
perform an action based on the steering information.

2. The system of claim 1, wherein the quantification task is a measurement of a blood flow parameter associated with the anatomical structure of interest.

3. The system of claim 1, wherein the quantification task is a measurement of a dimension of the anatomical structure of interest.

4. The system of claim 1, wherein the action is displaying steering guidance information that guides a user to steer the ultrasound catheter from the current orientation to the target orientation.

5. The system of claim 1, wherein the action is providing the steering information to a robotic surgical system.

6. The system of claim 1, wherein the action is displaying steering guidance information that includes a visual indicator that identifies an amount of movement of a steering component of the ultrasound catheter.

7. The system of claim 1, wherein the action is displaying steering guidance information that includes a set of visual indicators that respectively identify amounts of movement of a set of steering components of the ultrasound catheter.

8. A method comprising:
receiving a selection of an anatomical structure of interest of a subject and a quantification task associated with the anatomical structure of interest of the subject;
determining a current orientation of an ultrasound catheter in the subject;
determining a target orientation of the ultrasound catheter in the subject for the quantification task associated with the anatomical structure of interest of the subject;
determining steering information for steering the ultrasound catheter in the subject from the current orientation to the target orientation; and
performing an action based on the steering information.

9. The method of claim 8, wherein the quantification task is a measurement of a blood flow parameter associated with the anatomical structure of interest.

10. The method of claim 8, wherein the quantification task is a measurement of a dimension of the anatomical structure of interest.

11. The method of claim 8, wherein the action is displaying steering guidance information that guides a user to steer the ultrasound catheter from the current orientation to the target orientation.

12. The method of claim 8, wherein the action is providing the steering information to a robotic surgical system.

13. The method of claim 8, wherein the action is displaying steering guidance information that includes a visual indicator that identifies an amount of movement of a steering component of the ultrasound catheter.

14. The method of claim 8, wherein the action is displaying steering guidance information that includes a set of visual indicators that respectively identify amounts of movement of a set of steering components of the ultrasound catheter.

15. A non-transitory computer-readable medium storing instructions that, when executed by one or more processors, cause the one or more processors to:
receive a selection of an anatomical structure of interest of a subject and a quantification task associated with the anatomical structure of interest of the subject;
determine a current orientation of an ultrasound catheter in the subject;
determine a target orientation of the ultrasound catheter in the subject for the quantification task associated with the anatomical structure of interest of the subject;
determine steering information for steering the ultrasound catheter in the subject from the current orientation to the target orientation; and
perform an action based on the steering information.

16. The non-transitory computer-readable medium of claim 15, wherein the quantification task is a measurement of a blood flow parameter associated with the anatomical structure of interest.

17. The non-transitory computer-readable medium of claim 15, wherein the quantification task is a measurement of a dimension of the anatomical structure of interest.

18. The non-transitory computer-readable medium of claim 15, wherein the action is displaying steering guidance information that guides a user to steer the ultrasound catheter from the current orientation to the target orientation.

19. The non-transitory computer-readable medium of claim 15, wherein the action is providing the steering information to a robotic surgical system.

20. The non-transitory computer-readable medium of claim 15, wherein the action is displaying steering guidance information that includes a visual indicator that identifies an amount of movement of a steering component of the ultrasound catheter.
